(19) United States
(12) Patent Application Publication
(10) Pub. No.: US 2007/0142807 A1
(43) Pub. Date: Jun. 21, 2007

(54) Liquid absorbing cooling pad

(76) Inventors: Janet Armstrong Lee, Norcross, GA (US); Robert Eugene Lee, Norcross, GA (US)

Correspondence Address: LAURENCE T. PEARSON, 5503 HIGHLAND COURT, WILMINGTON, DE, 19802, US

(21) Appl. No.: 11/313,093

(22) Filed: Dec. 21, 2005

(51) Int. Cl. A61F 13/15

(52) U.S. Cl. 604/385.01; 604/385.06; 604/364

(57) ABSTRACT

An article for absorbing fluid from the perineum of a woman and simultaneously cooling the perineum. The article comprises two regions, said regions being a front region that contacts the woman's body over at least a portion of its surface, a rear region and an optional central fluid impermeable partition disposed between the front region and the rear region. A pocket between the regions contains a cooling medium.

### FIELD OF THE INVENTION

This invention relates to medical devices and more particularly to improvements in absorbent articles designed for single patient use.

### BACKGROUND ART

Ice and heat packs are known in the prior art. Both devices are known to assist in medical care for bruises, cuts, swelled joints, muscular strain and the like. For instance, it is known that the application of heat assists in muscular strain once swelling has reduced at the point of strain. Accordingly, hot water bottles can be used to apply heat directly to the muscular strained area. U.S. Pat. Nos. 1,711,876 and 1,819,913 both describe hot water bottle devices which can be used for heat treatment. Hot water is poured into these devices and closed, whereafter the device can be applied directly to a muscular strain.

At least one prior art invention suggests a device which can accommodate both heat or ice. In particular, U.S. Pat. No. 1,927,751 discloses a cover for a hot water bag or ice bag. This device employs straps which can be used to secure the device to a patient.

The use of ice to reduce swelling and inflammation of a wounded body area of a person is known to assist in the healing of that area. When trauma inflicts a body part, such as a knee, for example, swelling and inflammation of that area can occur. Inflammation is the result of the body introducing additional blood flow to the traumatized area. Additional blood flow assists in the healing of the wounded area by carrying away damaged or dead tissue. Swelling is the body's way of providing a “natural splint” to the traumatized area. Unfortunately both inflammation and swelling can cause additional pain to the person due to the force exerted upon the traumatized area. It is therefore advantageous to reduce the swelling and inflammation, and hence the need to apply ice to the inflicted body part.

Devices to assist in the reduction of inflammation and swelling are known. For instance, U.S. Pat. No. 4,628,932 describes an ice pack for use on a person's knee. Two compartments are employed to receive the ice. This device is helpful in the reduction of inflammation and swelling to a person's knee but is unfortunately limited in many ways. For instance, the device is limited for use on joints such as knees and elbows and lacks the structural components to be adaptable to other body parts. Further, it lacks an outer layer which could be used to reduce the temperature of the outside of the bag, which would make the bag more comfortable to hold by a person, and further lacks an absorbable material layer which could soak-up body fluids which may seep from the wounded area. This device could be wrapped in a cloth towel. However, if any body fluids seep from the wound (i.e., blood), the cloth towel would then need to be thrown away. This results in added expense and waste of a perfectly good towel. A person could instead wrap paper towels around the bag, but this too can add expense. Further, paper towels typically do not provide amble resistance from the coldness of the ice pack.

U.S. Pat. No. 4,951,666 describes an ice pack wrapable about a limb. The ice pack comprises a cloth outer bag having an exterior, an interior and an open top. A sealable plastic inner bag is received within the outer bag, the inner bag having an exterior formed by opposing planar portions, an interior and a mouth opening for receiving ice, the inner bag being foldable between a retracted position wholly contained within the interior of the outer bag and an extended position in which the inner bag protrudes from the open top of the outer bag for filling.

Some inventions have attempted to add an outer layer to their respective ice bag or heat pack. Such can be seen in U.S. Pat. Nos. 5,074,300, 5,133,348 and 5,456,704. Unfortunately, all of these inventions fall short of disclosing, let alone teach or suggest, a disposable ice pack having a fluid absorbable outer layer which soaks-up any body fluids of the wound and at the same time permits a person to hold the ice pack to the wound without being uncomfortable to hold due to the coldness of the ice pack. Such a device is clearly needed to overcome all of the deficiencies of the prior art.

The use of cold packs that do not use ice for thermal therapy is known. One type of cold pack contains an insulating material which, upon cooling in a refrigerator or freezer, gradually warms back to ambient temperature while being applied to a wounded body part. Another type of cold pack operates via a change of phase of the components of the cold pack. Still another type of cold pack employs chemical components that are endotherm-producing on dissolution in a solvent.

Phase change materials may be converted between solid and liquid phases and utilize a latent heat of fusion to absorb, store and release heat, or cool, during such phase conversion. Accordingly, the amount of energy absorbed upon melting or released upon freezing is greater than the amount of energy absorbed or released upon increasing or decreasing the temperature of the material. Ice/water is one example of a phase change material.

Certain chemical compounds, once dissolved into a solution, result in a lowering of the temperature of the solution below ambient temperature. On dissolution, these compounds absorb heat from the surrounding environment. For example, inorganic salts or soluble organic compounds known to have a positive enthalpy of aqueous solution are used to make the reduced temperature solutions useful in cold packs.

Examples of cold packs that employ an insulating material are cold packs that contain a gel. Typically, these cold packs are cooled in a refrigerator or freezer. For cold therapy, once cooled, the cold pack is placed on the injured or sore area and thus provides the cold therapy.

Chemical cold packs that provide an insulating layer between the cold pack and the skin are also known. One such cold pack uses an outer pouch containing capillaries to allow drainage of the pack and to provide a temperature moderating effect. Another chemical cold pack uses a wetting member in contact with the chemical cold pack, thereby providing moist cold to an injury. Yet another chemical cold pack positions an absorbent/insulating layer between the chemical cold and the ambient air with the chemical cold pack placed up against the patient's skin. In this device, the surface area of the absorbent/insulating layer is smaller than the surface area of the cold pack. One prior art chemical cold pack cover provides means to attach the cold pack to a patient and to provide a water-resistant material in contact with the skin.

U.S. Pat. No. 6,470,705 describes a disposable ice pack for receiving and retaining a frozen material and for compressing against an area of a person's body that has been traumatized. The disposable ice pack consists of a square bag portion having four side edges, including a sealable open top end, a closed bottom end and a pair of opposed side edges forming an inner cavity. A Ziplock® type closure mechanism is disposed along inner surfaces of the top end providing a water tight seal to the ice pack. A sheet material consisting of one or two layers of fluid absorbable cloth material is attached by heat sealing and juxtaposed to one of the bag portion outer walls. The fluid absorbable material makes contact with the person's traumatized body area and absorbs any body fluids seeping therefrom. The inner cavity receiving and retaining the frozen material.

U.S. Pat. No. 6,251,131 describes an absorbent ice bag comprising an ice bag defining a single-side surface area, and a resealable opening. An absorbent article which may be hour glass shaped for use over the perineum is attached to the ice bag, the absorbent article defines an area such that the total area of the article is larger than the single side-surface area. A phase change material may be added to the ice bag through a resealable opening and wherein the unobstructed perimeter area provides an unobstructed area for absorbing a bodily fluid on the absorbent article.

One disadvantage of the product of the '131 patent is that there is a reduced absorbency in the region where there is ice bag and absorbent pad.

One problem that is not addressed effectively by any of the forementioned is the need to apply cold to a female patient postpartum who has given birth vaginally. Such patients require a comfortable fitting shaped pad with effective absorbency and the ability to apply cold to the same area from which bodily fluids are discharged. When ice is used as the cooling medium, the pad must remain dry.

The present invention addresses these needs by providing for an absorbent pad that also applies cold to the perineal area and in which the absorbency of the pad is not affected by the presence of an ice bag.

### SUMMARY

The present invention is directed towards an article for absorbing fluid from the perineal area of a woman and simultaneously cooling the perineum. The article comprises a sealed front region that contacts the woman's body over at least a portion of its surface and absorbs bodily fluids from the woman, a sealed rear region and an optional central fluid impermeable partition disposed between the front region and the rear region and attached to the front region. The front region comprises a body facing first fluid permeable web and a first absorbent web. The rear region further comprises a second absorbent web and a fluid impermeable backsheet adjacent to the second absorbent web on the opposite surface to the fluid permeable web,

The front and rear regions are sealed around their edges and are separated by a pocket for containing a cooling medium, said pocket being formed by sealing around the edges of the front and rear regions such that a gap exists through which a cooling medium can be added into the pocket, and such that the edges of the front and rear regions adjacent to the gap comprise a means for sealing the gap to contain the cooling medium in the pocket.

In a further embodiment of the invention the body facing first fluid permeable web is a non woven or a formed or apertured film.

In a still further embodiment of the invention the body facing first absorbent web comprises a non woven which optionally comprises a superabsorbent polymer.

The article of the invention can further comprise a fluid distribution layer between the first fluid permeable web and the first absorbent web.

In a still further embodiment of the invention the second absorbent web comprises a non woven and optionally comprises a superabsorbent polymer.

In a still further embodiment of the invention a second fluid permeable web is located between the pocket and the second absorbent web and in which the second fluid permeable web comprises a formed film or a non woven material that is optionally joined by a portion of its edge to the first fluid permeable web and the fluid impermeable backsheet.

In a still further embodiment of the invention the optional fluid impermeable partition comprises a polyolefin film.

In a still further embodiment of the invention the fluid impermeable backsheet comprises a polyolefin film.

The cooling medium is ice in one embodiment of the invention.

In a still further embodiment of the invention is shaped as an hourglass with the narrow portion of the hourglass intended to be situation between the wearer's legs and over the perineal area. In a still further embodiment of the invention the sides are elasticated.

In a still further embodiment of the invention the fluid impermeable backsheet is permeable to vapor and gas but not to liquid.

In a still further embodiment of the invention the means for sealing is selected from the group consisting of a Ziplock® fastener, Velcro®, and a pressure sensitive adhesive.

In a still further embodiment of the invention the first absorbent web, or the second absorbent web, or both are joined to the body facing first fluid permeable web, the fluid impermeable backsheet, or the optional central fluid impermeable partition by their edges or any portion of their surfaces.

The fluid impermeable backsheet, and the optional central fluid impermeable partition are joined by adhesives around a portion of their edges. Alternatively in the article of the invention, the body facing first fluid permeable web, the fluid impermeable backsheet, and the optional central fluid impermeable partition are joined by stitching around a portion of their edges.

The fluid impermeable backsheet can also comprise adhesive on all or a portion of its external surface, the external surface being that which faces away from the interior of the article.

### BRIEF DESCRIPTION OF THE DRAWINGS

FIG. 1 shows a cross section of the layer arrangement of an embodiment of the product of the invention.

### DETAILED DESCRIPTION OF THE INVENTION

### Definitions

The term “film” refers to a web made by extruding a molten sheet of thermoplastic polymeric material by a cast or blown extrusion process and then cooling the sheet to form a solid polymeric web. Films can be monolayer films, coextruded films, coated films, and composite films. Coated films are films comprising a monolayer or coextruded film which are subsequently coated (for example, extrusion coated, impression coated, or printed) with a thin layer of the same or different material to which it is bonded and after bonding is incapable of separation. Composite films are films comprising more than one film where at least two films are combined in a bonding process. Bonding processes may incorporate adhesive layers between the film layers. Films also denote cast films that are not made using an extrusion process.

Throughout this description, the expression “apertured film” denotes a film having a plurality of holes that extend from one surface to a second surface. A two dimensional apertured film is a film in which no three dimensional structure exists in or around the holes, which then connect the second surface of a flat film to the first surface of the film. A three dimensional film is a film with protuberances or other dimensional structures.

By “formed film” is meant films made by forming two-dimensional sheets to create three-dimensional formed films. It is known to vacuum-form two-dimensional sheets to create three-dimensional formed films, by causing a softened or molten polymer sheet to conform to the shape of a forming screen. Commonly utilized apertured formed film topsheets are disclosed in, for example U.S. Pat. Nos. 3,929,135, 4,324,246, 4,342,314, 4,252,516, 4,535,020, 5,591,510, and U.S. patent application Ser. No. 10/082,040 filed Feb. 20, 2002. The formed films described in these publications are formed by application of a fluid pressure differential where the fluid preferably is air to thereby cause the polymer sheet to conform to the screen and also to cool. A preferred feature of these formed films is that the polymer crystallizes, or “sets” after the molten polymer has been formed. The resultant formed film therefore is said to have “memory.” Memory enables the formed film to regain its original form after non-deforming forces are applied to it, for example while the film is stored in the form of a wound roll. A disadvantage of vacuum formed films is that the body contacting surface of the film my have a plastic feeling that can feel both sticky and hot to the wearer of the article.

It also is known to utilize high pressure water jet systems, known as hydroforming systems, to cause two-dimensional polymeric films to conform to forming screens in order to create three-dimensional films. U.S. Pat. Nos. 4,609,518, 4,629,643, 4,695,422, 4,778,644, and 4,839,216, all to Curro (referred to herein as “Curro”) and assigned to Procter & Gamble, and U.S. Pat. Nos. 5,770,144, 6,022,607, and 6,240,817 assigned to McNeil, describe a multitude of film designs created by the combination of forming screens using high pressure water jet systems. In these systems, pressurized water is ejected from nozzles projecting water onto the surface of a polymeric film causing it to conform to the surface of an underlying support screen. The screen has indentations or perforations disposed on its surface. As the screen rotates it causes subsequent portions of the film to pass under the water jet causing subsequent portions of the film to conform to the screen. An advantage of this method of making formed films is that the number of protrusions that can be made in a specific surface area of film is greater than what is possible in a vacuum formed film process. Another advantage is that by utilizing water to deform crystalline film high levels of energy are applied to the film thereby causing more work and making the film softer.

The term “web” refers to a material capable of being wound into a roll. Webs can be film webs, nonwoven webs, woven webs, laminate webs, apertured laminate webs etc.

The term “nonwoven” means a web including a multitude of fibers. The fibers can be bonded to each other or can be unbonded. The fibers can be staple fibers or continuous fibers. The fibers can comprise a single material or a multitude of materials, either as a combination of different fibers or as a combination of similar fibers each comprised of different materials.

A nonwoven fibrous web useful in embodiments of the invention may comprise fibers of polyethylene, polypropylene, elastomers, polyesters, rayon, cellulose, nylon, and blends of such fibers. A number of definitions have been proposed for nonwoven fibrous webs. The fibers usually include staple fibers or continuous filaments. As used herein “nonwoven fibrous web” is used in its generic sense to define a generally planar structure that is relatively flat, flexible and porous, and is composed of staple fibers or continuous filaments. For a detailed description of nonwovens, see “Nonwoven Fabric Primer and Reference Sampler” by E. A. Vaughn, ASSOCIATION OF THE NONWOVEN FABRICS INDUSTRY, 3d Edition (1992). The nonwovens may be carded, spun bonded, wet laid, air laid and melt blown as such products are well known in the trade.

The nonwoven web of the present invention can be the product of any process for forming the same. Examples of methods for manufacturing non woven webs that are well known to those skilled in the art are the processes that produce spunbond and melt blown non woven webs. The non woven web of which embodiments of this invention comprise can also be a composite or combination of webs, such as spunbond or melt blown webs. In a preferred embodiment of the invention, the web is a spunbond material made of polypropylene fiber. However, the non woven web can comprise any polymeric material from which a fiber can be produced.

“Superabsorbent polymers” refers to synthetic cross-linked polymeric materials that are capable of absorbing many times their own weight in water and other liquids. As used herein, the term “SAP” means a superabsorbent polymer which, when in a substantially dry state, has the ability to spontaneously imbibe more than (20) times its own weight in aqueous fluid, for example, tap water. Any superabsorbent polymer (SAP) can be used in the present invention. Usually, the SAP component is a cellulosic-derived particle, polyacrylic acid based material, and the like. Typically, SAP is produced in granular form, such granules exhibiting a particle size distribution and an average particle size distribution. The average particle size distribution should be related to the un-stretched thickness of the film in such way that the largest particles fit within the thickness of the un-stretched films. Particles produced in larger than adequate sizes may be ground down to the appropriate size, if needed.

By “means for sealing” is meant the means by which a pouch or bag that the invention comprises for holding a cooling medium can be sealed around all or a portion of its edge to make if fluid tight. Ziploc®, Velcro® (Velcro Industries B.V., Curacao, NL), or adhesive on the surfaces of the pad, said adhesive sealing when pressed with the force applied by a human hand. Velcro® can be applied to two surfaces to be sealed or to a flap on one surface that folds over to attach to a Velcro® strip attached to a second surface.

In one embodiment of the invention the sides of the pad are elasticated to ensure a closer fit to the woman's body. Any elastic material maybe used, but preferably the material will be either a metallocene based low density polyethylene (m-LDPE), or a block-copolymer blend that contains styrene/butadiene/styrene (SBS), styrene/ethylene-butylene/styrene (SEBS), ethylene vinyl acetate (EVA), thermoplastic urethane, or cross-linked rubber. Desirably, the elastic polymeric film has a basis weight of from about 18 g/m² to about 100 g/m². Preferably, an m-LDPE film has a basis weight of about 25 g/m², whereas block copolymer films have a basis weight of about 50 g/m². Also, it is desirable that the elastic polymeric files have less than 25% set when stretched 50%.

Elastication can be accomplished by making one of the layers of the pad of elastic material, or by attaching an elastic strip to the edge of the pad by adhesive or some other means for attachment known to one skilled in the art.

By “cooling medium” is meant any solid or fluid that has sufficient heat capacity to be used in the product of the invention to take heat form the patient's body. In one embodiment of the invention the preferred cooling medium is ice, due to its availability and low cost. The use of cold packs that do not use ice for thermal therapy is known and one skilled in the art will be able to select a suitable cooling medium for use in the invention.

One type of cooling medium contains an insulating material which, upon cooling in a refrigerator or freezer, gradually warms back to ambient temperature while being applied to an wounded body part. Another type of cold pack operates via a change of phase of the components of the cold pack. Still another type of cold pack employs chemical components that are endotherm-producing on dissolution in a solvent.

Phase change materials as cooling media may be converted between solid and liquid phases and utilize a latent heat of fusion to absorb, store and release heat, or cool, during such phase conversion. Accordingly, the amount of energy absorbed upon melting or released upon freezing is greater than the amount of energy absorbed or released upon increasing or decreasing the temperature of the material. Ice/water is one example of a phase change material.

Chemical compounds that, once dissolved into a solution, result in a lowering of the temperature of the solution below ambient temperature. On dissolution, these compounds absorb heat from the surrounding environment. For example, inorganic salts or soluble organic compounds known to have a positive enthalpy of aqueous solution are used to make the reduced temperature solutions useful as cooling media.

Examples of cold packs that employ an insulating material are cold packs that contain a gel. Typically, these cold packs are cooled in a refrigerator or freezer. For cold therapy, once cooled, the cold pack is placed on the injured or sore area and thus provides the cold therapy.

The article can be assembled by any means known to one skilled in the art and the edges of selected sheets that the pad comprises adhered to each other around a margin adjacent to the edges of the sheets. Means for adhesion can include hot melt or solvent based adhesives. In non limiting examples, polymers that have been used in hot melt adhesives employed in the construction of disposable nonwoven goods have included S-I-S (styrene-isoprene-styrene); SBS (styrene-butadiene-styrene); SEBS (styrene-ethylene-butylene-styrene); EVA (ethylene vinyl acetate); and APAO (amorphous poly alpha olefin).

The sheets that the article comprises can also be assembled by stitching around the edges of the sheets.

### Preferred Embodiments

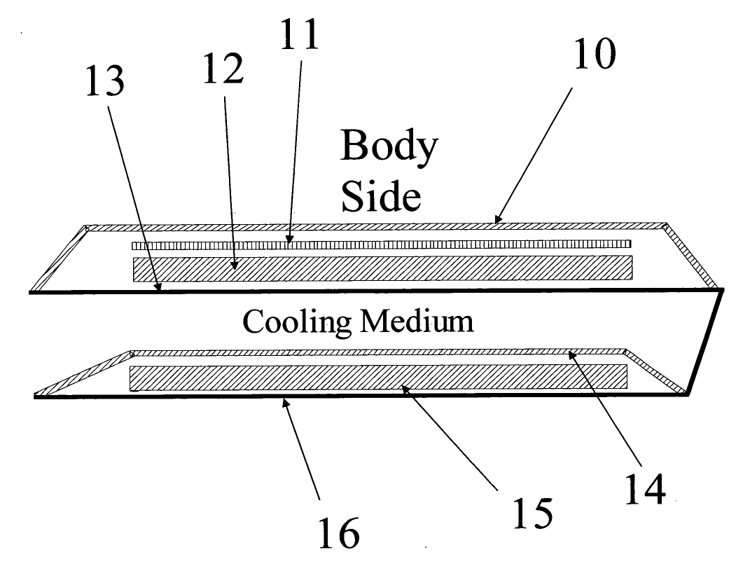

Referring now to FIG. 1, the figure shows a cross section of the arrangement of layers in an embodiment of the invention. A topsheet (10) which is fluid permeable and through which the bodily fluids pass, is located on the body side of the product. The topsheet can comprise a non woven or a formed film or some combination of the two. In a preferred embodiment of the invention the topsheet comprises a spunbond non woven comprising polypropylene fiber.

The embodiment further comprises an optional fluid distribution layer (11). A non limiting example of such a layer is found in U.S. Pat. No. 6,700,036 to Tredegar. The function of the layer is to distribute fluid in the plane of the article before it impinges on the absorbent layer.

In all embodiments the product will comprise a first absorbent web (12) whose function is to absorb bodily fluids arriving from the topsheet (10). The first absorbent web can be any material that absorbs fluid, but in a preferred embodiment comprises a non woven pad, optionally containing a superabsorbent polymer. In all embodiments the product will also comprise a second absorbent web (15) whose function is to absorb melting heat transfer material, such as water from ice. The absorbent webs can independently be any material that absorbs fluid. For example, either absorbent core can be a fluffy batt cut from a relatively loose web of non-woven fibers having a relatively high absorptive capacity. The absorbent cores usually have a rectangular configuration, and may optionally have inwardly curved side edges, such as an hourglass shape. The absorbent core is usually smaller in surface area than the backing sheet and the cover. The absorbent core may also be a fibrous batt having an integral densified layer. In such a case, the absorbent core is positioned on the backing sheet of the absorbent article so that the densified layer adjoins the backing sheet. The densified layer has relatively higher wettability and liquid retentivity than the rest of the aforementioned batt and usually is formed by slightly moistening one surface of the batt and thereafter compressing the moistened surface.

The absorbent core may contain any material that absorbs bodily secretions including, but not limited to pulp, polymeric fibers and filaments, spagnum moss, natural fibers, superabsorbent polymers (including fibers, particulate material and foams), absorbent foams, and other such absorbent materials. The absorbent core can comprise cellulosic fibers and superabsorbent polymer particles. The absorbent core may also include additional materials such as odor control material, wetness indicator material, materials for administering or delivering medicaments, such as encapsulated medicaments, and materials for maintaining skin moisture, such as encapsulated moisturizers.

The invention also comprises a liquid impermeable backsheet (16) and an optional fluid impermeable sheet (13) that optionally separates the first absorbent core (12) from the second absorbent core (15) and a permeable sheet (14) that may optionally be integrated into he second absorbent core (15). The permeable sheet (14) can comprise a non woven or a formed film or some combination of the two. In a preferred embodiment of the invention the topsheet comprises a spunbond non woven comprising polypropylene fiber.

The liquid impermeable backsheet (16) can also comprise an adhesive on its outward facing side that covers all or a part of the area of the backsheet and is suitable for adhering the article to cloth, for example the user's underwear, and prevents the article from moving relative to the cloth.

The topsheet (10) and fluid impermeable sheets (13 and 16) are usually substantially coextensive and are joined together about the periphery of the absorbent article, which may be hourglass shaped to allow for comfortable fit to the perineum of a patient. The seal between the topsheet (10) and the backsheet (16) leaves open a portion of the edge so that a cooling medium can be inserted into the article and the article then sealed my a means for sealing as known to one skilled in the art.

The front region of the article is defined by topsheet (10), optional fluid distribution layer (11), the first absorbent core (12) and optional fluid impermeable sheet (13), which are sealed around their edges to form a liquid tight compartment. Similarly, the rear region is formed by the fluid permeable cheet (14), the second absorbent core (15) and the fluid impermeable backsheet (16) which are in turn all sealed around their edges to form a liquid impermeable region. The front and rear regions are then sealed around a portion of their edges sufficient to leave a gap through which cooling medium can be inserted. The gap can be sealed by a means for sealing as described supra.

The absorbent cores (12) and (15) may be anchored to their respective covers (10, 11 and 14), or to the backsheet or other components of the absorbent article. Methods for attaching the components together are included supra in the discussion of adhesives. A sealable gap is left between the front portion of the article as defined by items 10, 11, 12, and optional 13, and the rear portion as defined by itens 14, 15, and 16. This gap is large enough to insert ice, and can be sealed by any means for sealing as described supra in order to stop cooling medium form leaking from the article.

The impermeable sheets (13 and 16) may be of any flexible material that prevents the transfer of fluid through it, but does not necessarily prevent the passages of gases. Sheets that are pervious to vapor are known as breathable sheets. In general, these backsheets are intended to allow the passage of vapor through them while retarding, at least to a degree, the passage of fluid. Porous film technology provides materials that can be used to form sheets that allow vapor transmission, but are relatively impervious to liquids. Commonly used materials are polyethylene or polypropylene films. Other materials that may be used as impermeable barriers may be chosen from films of polyesters, polyamides, polyethylene vinyl acetate, polyvinyl chloride, and polyvinylidene chloride. Co-extruded and laminated combinations of the foregoing, wherein such combinations are permitted by the chemical and physical properties of the film, may be used. Fluid impermeable nonreticulated foams and repellent treated papers may also be used. Films that are fluid barriers, but permit gases to transpire, i.e., “breathable films,” may also be used.

The invention has been described here in terms of certain embodiments, but it is to be understood that any variations on these embodiments that are apparent to one skilled in the art are also included in the scope of the claims. All references cited herein are hereby incorporated herein in their entirety.

I claim:

1.) An article for absorbing fluid from the perineal area of a woman and simultaneously cooling the perineum, said article comprising a sealed front region that contacts the woman's body over at least a portion of its surface and absorbs bodily fluids from the woman, a sealed rear region and an optional central fluid impermeable partition disposed between the front region and the rear region and attached to the front region, in which article the front region comprises
(i) a body facing first fluid permeable web, and
(ii) a first absorbent web,
said rear region further comprising
(iii) a second absorbent web
(iv) a fluid impermeable backsheet adjacent to the second absorbent web
on the opposite surface to the fluid permeable web,
in which the front and rear regions are sealed around their edges and are separated by a pocket for containing a cooling medium, said pocket being formed by sealing around the edges of the front and rear regions such that a gap exists through which a cooling medium can be added into the pocket, and such that the edges of the front and rear regions adjacent to the gap comprise a means for sealing the gap to contain the cooling medium in the pocket.

2.) The article of claim 1 in which the body facing first fluid permeable web comprises a non woven.

3.) The article of claim 1 in which the body facing first fluid permeable web comprises a formed film

4.) The article of claim 1 in which the body facing first absorbent web comprises a non woven.

5.) The article of claim 1 in which the body facing first absorbent web comprises a superabsorbent polymer.

6.) The article of claim 1 which further comprises a fluid distribution layer between the first fluid permeable web and the first absorbent web.

7.) The article of claim 1 in which the second absorbent web comprises a non woven.

8.) The article of claim 1 in which the second absorbent web comprises a superabsorbent polymer.

9.) The article of claim 1 in which a second fluid permeable web is located between the pocket and the second absorbent web and is attached to the rear region, and in which the second fluid permeable web comprises a formed film or a non woven material.

10.) The article of claim 1 in which the optional fluid impermeable partition comprises a polyolefin film.

11.) The article of claim 1 in which the fluid impermeable backsheet comprises a polyolefin film.

12.) The article of claim 1 in which the cooling medium is ice.

13.) The article of claim 1 which is shaped as an hourglass.

14.) The article of claim 1 with an edge that is elasticated around all or a portion of the edge.

15.) The article of claim 1 in which the fluid impermeable backsheet is permeable to vapor and gas but not to liquid.

16.) The article of claim 1 in which the means for sealing is selected from the group consisting of a Ziplock® fastener, Velcro®, and a pressure sensitive adhesive.

17.) The article of claim 1 in which the first absorbent web, or the second absorbent web, or both are joined to the body facing first fluid permeable web, the fluid impermeable backsheet, or the optional central fluid impermeable partition by their edges or any portion of their surfaces.

18.) The article of claim 1 in which the body facing first fluid permeable web, the fluid impermeable backsheet, and the optional central fluid impermeable partition are joined by adhesives around a portion of their edges.

19.) The article of claim 1 in which the body facing first fluid permeable web, the fluid impermeable backsheet, and the optional central fluid impermeable partition are joined by stitching around a portion of their edges.

20.) The article of claim 1 in which the fluid impermeable backsheet, comprises adhesive on all or a portion of its external surface, the external surface being that which faces away form the interior of the article.
